(19) United States
(12) Patent Application Publication
(10) Pub. No.: US 2024/0226571 A1
(43) Pub. Date: Jul. 11, 2024

(54) MEDICAL DEVICE SYSTEMS FOR PROVIDING IMPEDANCE STATUS

(71) Applicant: Shiratronics, Inc., Brooklyn Park, MN (US)

(72) Inventor: Mitchell A. Tramp, Corcoran, MN (US)

(21) Appl. No.: 18/405,230

(22) Filed: Jan. 5, 2024

Related U.S. Application Data
(60) Provisional application No. 63/438,312, filed on Jan. 11, 2023.

(51) Int. Cl. A61N 1/36; A61N 1/02; A61N 1/372

(52) U.S. Cl. CPC A61N 1/3614; A61N 1/025; A61N 1/36185; A61N 1/37247; A61N 1/37252

(57) ABSTRACT

A system may include a medical device and at least one external device. The medical device may have a plurality of electrodes, and may be configured to measure impedance for each of the plurality of electrodes and generate impedance data. The eternal device(s) may include a display and a processor. The medical device may be configured to communicate the generated impedance data to the at least one eternal device. The processor may be configured to provide on the display a plurality of electrode icons corresponding to the plurality of electrodes, to determine, based on the impedance data, one or more impedance state electrodes from the plurality of electrodes that have an impedance state, and to provide on the display an impedance status representation for one or more of the plurality of electrode icons corresponding to the one or more impedance state electrodes.

### PRIORITY

This application claims the benefit of priority to U.S. Provisional Patent Application Ser. No. 63/438,312, filed Jan. 11, 2023 which is incorporated by reference herein in its entirety.

### TECHNICAL FIELD

This document relates generally to medical systems, and more particularly, but not by way of limitation, medical device systems and methods for providing electrode impedance status.

### BACKGROUND

Medical devices may sense electrical signals and/or deliver an electrical therapy. For example, medical devices may include implantable devices configured to deliver a therapy. Implantable neurostimulators are a specific example of implantable electrical therapy devices. A fully head-located implantable peripheral neurostimulation system, having one or more implantable devices, designed for the treatment of chronic head pain is a specific example of an implantable neurostimulation system.

It is desirable to measure electrode impedance, as variations in electrode impedance that exceeds an expected range may adversely affect the ability to deliver therapy or sense electrical signals. Furthermore, some medical devices may include more than one lead with more than one electrode on each lead. These systems have more electrode impedances to measure, which can make it overwhelming for a user of the system to monitor or program the system.

### SUMMARY

An example (e.g., “Example 1”) of a system may include a medical device and at least one external device. The medical device may have a plurality of electrodes, and may be configured to measure impedance for each of the plurality of electrodes and generate impedance data. The eternal device(s) may include a display and a processor. The medical device may be configured to communicate the generated impedance data to the at least one eternal device. The processor may be configured to provide on the display a plurality of electrode icons corresponding to the plurality of electrodes, to determine, based on the impedance data, one or more impedance state electrodes from the plurality of electrodes that have an impedance state, and to provide on the display an impedance status representation for one or more of the plurality of electrode icons corresponding to the one or more impedance state electrodes.

In Example 2, the subject matter of Example 1 may optionally be configured such that the processor is configured to provide on the display a therapy programming display screen, and receive user input via the therapy programming display screen to program an electrical therapy including to select active ones of the plurality of electrodes for use to deliver the electrical therapy. The impedance status representation may be on the therapy display screen used to program the electrical therapy.

In Example 3, the subject matter of Example 2 may optionally be configured such that the impedance status representation includes an “X” on or near the one or more of the plurality of icons corresponding to the one or more impedance state electrodes.

In Example 4, the subject matter of any one or more of Examples 1-3 may optionally be configured such that the impedance status representation on the display does not include an impedance measurement.

In Example 5, the subject matter of any one or more of Examples 1-4 may optionally be configured such that the one or more impedance state electrodes have an out-of-bounds impedance.

In Example 6, the subject matter of any one or more of Examples 1-4 may optionally be configured such that the one or more impedance state electrodes have a valid impedance.

In Example 7, the subject matter of any one or more of Examples 1-6 may optionally be configured such that the plurality of electrodes is on a lead, and the processor is configured to provide on the display a representation of the lead with the plurality of electrode icons.

In Example 8, the subject matter of Example 7 may optionally be configured such that each of the electrode icons is labeled with a corresponding electrode number.

In Example 9, the subject matter of any one or more of Examples 1-8 may optionally be configured such that the impedance status representation includes a color identifying the one or more impedance state electrodes.

In Example 10, the subject matter of any one or more of Examples 1-9 may optionally be configured such that the impedance status representation includes a line segment under the one or more of the plurality of icons corresponding to the one or more impedance state electrodes.

In Example 11, the subject matter of any one or more of Examples 1-10 may optionally be configured such that the impedance status representation includes an “X” on or near the one or more of the plurality of icons corresponding to the one or more impedance state electrodes.

In Example 12, the subject matter of any one or more of Examples 1-11 may optionally be configured such that the impedance status representation is a first impedance status representation, the processor is configured to determine, based on the impedance data, one or more second impedance state electrodes from the plurality of electrodes, and the processor is configured to provide on the display a second impedance status representation for one or more of the plurality of electrode icons corresponding to the one or more second impedance state electrodes.

In Example 13, the subject matter of any one or more of Examples 1-12 may optionally be configured such that the medical device is configured deliver electrical stimulation using the plurality of electrodes.

In Example 14, the subject matter of any one or more of Examples 1-13 may optionally be configured such that the medical device is configured to sense electrical signals using the plurality of electrodes.

In Example 15, the subject matter of any one or more of Examples 1-14 may optionally be configured such that to provide on the display an impedance detail icon, and to respond to selection of the impedance detail icon by displaying in a separate window the measured impedances for the plurality of electrodes.

In Example 16, the subject matter of any one or more of Examples 1-15 may optionally be configured such that the processor is configured to provide on the display a date and time when the impedance was measured.

In Example 17, the subject matter of Example 1 may optionally be configured such that the plurality of electrodes is on a lead, the processor is configured to provide on the display a representation of the lead with the plurality of electrode icons, each of the electrode icons are labeled with a corresponding electrode number, and the impedance status representation for the one or more of the plurality of icons includes at least one of: a line segment under the one or more of the plurality of icons corresponding to the one or more impedance state electrodes; an “X” on or near the one or more of the plurality of icons corresponding to the one or more impedance state electrodes; or a color associated with the one or more of the plurality of icons corresponding to the one or more impedance state electrodes.

In Example 18, the subject matter of Example 17 may optionally be configured such that the one or more impedance state electrodes have an out-of-bounds impedance, and the impedance status representation for the one or more of the plurality of icons indicates that the corresponding one or more impedance electrodes have the out-of-bounds impedance.

In Example 19, the subject matter of Example 17 may optionally be configured such that the one or more impedance state electrodes have a valid impedance, and the impedance status representation for the one or more of the plurality of icons indicates that the corresponding one or more impedance electrodes has the valid impedance.

In Example 20, the subject matter of Example 17 may optionally be configured such that the one or more impedance state electrodes include one or more first impedance state electrodes having an out-of-bounds impedance and one or more second impedance state electrodes having a valid impedance, and the impedance status representation includes a first impedance status representation indicating that the corresponding one or more first impedance state electrodes have the out-of-bounds impedance, and a second impedance status representation indicating that the corresponding one or more of the second impedance state electrodes have the valid impedance.

An example (e.g., “Example 21”) of subject matter (e.g., a method, a means for performing acts, or a machine-readable medium including instructions that, when performed by the machine, cause the machine to perform acts) may be performed using system that includes a medical device having a plurality of electrodes and at least one external device including a display and a processor. The subject matter may include using the medical device to measure impedance for each of the plurality of electrodes and generate impedance data, communicating the generated impedance data to the at least one eternal device, using the processor to determine, based on the impedance data, one or more impedance state electrodes from the plurality of electrodes that have an impedance state, and providing on the display a plurality of electrode icons corresponding to the plurality of electrodes, and an impedance status representation for one or more of the plurality of electrode icons corresponding to the one or more impedance state electrodes.

In Example 22, the subject matter of Example 21 may optionally be configured to further include providing on the display a therapy programming display screen, and receiving user input via the therapy programming display screen to program an electrical therapy including to select active ones of the plurality of electrodes for use to deliver the electrical therapy. The impedance status representation may be on the therapy display screen used to program the electrical therapy.

In Example 23, the subject matter of Example 22 may optionally be configured such that the impedance status representation includes an “X” on or near the one or more of the plurality of icons corresponding to the one or more impedance state electrodes.

In Example 24, the subject matter of any one or more of Examples 21-23 may optionally be configured such that the plurality of electrodes is on a lead, and the subject matter further includes providing on the display a representation of the lead with the plurality of electrode icons, and labeling each of the electrode icons with a corresponding electrode number, wherein the impedance status representation includes a line segment adjacent the corresponding electrode.

In Example 25, the subject matter of Example 24 may optionally be configured such that the line segment includes an identifying color.

In Example 26, the subject matter of any one or more of Examples 21-25 may optionally be configured such that the one or more impedance state electrodes have an out-of-bounds impedance.

In Example 27, the subject matter of any one or more of Examples 21-25 may optionally be configured such that the one or more impedance state electrodes have a valid impedance.

In Example 28, the subject matter of any one or more of Examples 21-25 may optionally be configured such that the impedance status representation is a first impedance status representation, and the subject matter further includes using the processor to determine, based on the impedance data, one or more second impedance state electrodes from the plurality of electrodes, and providing on the display a second impedance status representation for one or more of the plurality of electrode icons corresponding to the one or more second impedance state electrodes.

In Example 29, the subject matter of Example 28 may optionally be configured such that one of the first impedance state electrodes or the second impedance state electrodes have an output-of-bounds impedance, and the other one of the first impedance state electrodes or the second impedance state electrodes have a valid impedance.

An example (e.g., “Example 30”) of subject matter (e.g., a means for performing acts, or a machine-readable medium including instructions that, when performed by the machine, cause the machine to perform acts) may include providing on a display a plurality of electrode icons corresponding to a plurality of electrodes for a medical device, receiving impedance data for the plurality of electrodes, determining, based on the impedance data, one or more impedance state electrodes from the plurality of electrodes, and providing on the display an impedance status representation for one or more of the plurality of electrode icons corresponding to the one or more impedance state electrodes.

In Example 31, the subject matter of Example 30 may optionally be configured to further include providing on the display a therapy programming display screen, and receiving user input via the therapy programming display screen to program an electrical therapy including to select active ones of the plurality of electrodes for use to deliver the electrical therapy. The impedance status representation may be on the therapy display screen used to program the electrical therapy.

In Example 32, the subject matter of Example 31 may optionally be configured such that the impedance status representation includes an “X” on or near the one or more of the plurality of icons corresponding to the one or more impedance state electrodes.

In Example 33, the subject matter of any one or more of Examples 30-32 may optionally be configured such that the plurality of electrodes is on a lead, and the subject matter further includes providing on the display a representation of the lead with the plurality of electrode icons, and labeling each of the electrode icons with a corresponding electrode number, wherein the impedance status representation includes a line segment adjacent the corresponding electrode.

In Example 34, the subject matter of Example 33 may optionally be configured such that the line segment includes an identifying color.

In Example 35, the subject matter of any one or more of Examples 30-34 may optionally be configured such that the one or more impedance state electrodes have an out-of-bounds impedance.

In Example 36, the subject matter of any one or more of Examples 30-34 may optionally be configured such that the one or more impedance state electrodes have a valid impedance.

In Example 37, the subject matter of any one or more of Examples 30-36 may optionally be configured such that the impedance status representation is a first impedance status representation, and the subject matter further includes using the processor to determine, based on the impedance data, one or more second impedance state electrodes from the plurality of electrodes, and providing on the display a second impedance status representation for one or more of the plurality of electrode icons corresponding to the one or more second impedance state electrodes.

In Example 38, the subject matter of Example 37 may optionally be configured such that one of the first impedance state electrodes or the second impedance state electrodes have an output-of-bounds impedance, and the other one of the first impedance state electrodes or the second impedance state electrodes have a valid impedance.

In Example 39, the subject matter of any one or more of Examples 30-38 may optionally be configured to automatically lock out an electrode having an invalid impedance from being used in an electrical therapy program.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIGS. 1A-1C illustrate, by way of example and not limitation, various systems for providing electrode impedance status.

FIG. 2 illustrates, by way of example and not limitation, a method for providing electrode impedance status.

FIG. 3 illustrates, by way of example and not limitation, a method implemented by a machine (such as a programmer) for providing electrode impedance status.

FIG. 4 illustrates, by way of example and not limitation, a method that may be performed using the impedance status.

FIG. 5 illustrates an implantable medical device implanted beneath the skin and over a patient's cranium.

FIG. 6 illustrates an external device and headset configured for use to communicate with and/or charge the implantable medical device(s).

FIG. 7 illustrates, by way of example and not limitation, two implanted devices with leads to cover both sides of the head with one of the devices on the left side of the head and the other on the right side of the head, and a charging/communication headset disposed about the cranium.

FIG. 8 illustrates, by way of example and not limitation, a side view of fully head-located neurostimulation system.

FIG. 9 illustrates, by way of example, and not limitation, a display with electrode icons and some examples of impedance status representations for the electrode icons.

FIG. 10 illustrates, by way of example, and not limitation, a display with electrode icons and other examples of impedance status representations for the electrode icons.

FIG. 11 illustrates, by way of example and not limitation, a therapy programming display screen configured for use to program an electrical therapy including selecting active ones of the plurality of electrodes for use to deliver the electrical therapy, where the therapy programming display screen includes the impedance status representation.

FIG. 12 illustrates, by way of example and not limitation, a therapy overview screen with more than one therapy program and more than one lead for each program.

FIG. 13 illustrates, by way of example and not limitation, electrode impedance detail for one of the leads.

FIG. 14 illustrates, by way of example and not limitation, a programming editing screen.

FIG. 15 illustrates, by way of example and not limitation, an electrode impedance detail screen that may be entered by selecting the magnifying glass control in FIG. 14.

FIG. 16 illustrates, by way of example and not limitation, a therapy overview screen for a scenario in which there is an out-of-bounds result for an electrode that is not being used to deliver a therapy.

FIG. 17 illustrates, by way of example and not limitation, an electrode impedance detail screen for the Left Occipital lead illustrated in FIG. 16.

FIG. 18 illustrates, by way of example and not limitation, an electrode impedance detail screen for electrode 4 for the Left Occipital lead illustrated in FIG. 16.

FIG. 19 illustrates, by way of example and not limitation, an electrode impedance detail screen for electrode 5 for the Left Occipital lead illustrated in FIG. 16.

FIG. 20 illustrates, by way of example and not limitation, a program editing screen for the scenario in which there is an out-of-bounds result for an electrode that is not being used to deliver a therapy.

FIG. 21 illustrates, by way of example and not limitation, a therapy overview screen for a scenario in which there is an out-of-bounds result for an electrode that is being used to deliver a therapy as identified by the “X” representation in the left occipital lead card in both of the Program A Therapy Card and Program B Therapy Card.

FIG. 22 illustrates, by way of example and not limitation, an electrode impedance detail screen for the Left Occipital lead illustrated in FIG. 21.

FIG. 23 illustrates, by way of example and not limitation, an electrode impedance detail screen for the Left Occipital lead illustrated in FIG. 21.

FIG. 24 illustrates, by way of example and not limitation, an electrode impedance detail screen for the Left Occipital lead illustrated in FIG. 21.

FIG. 25 illustrates, by way of example and not limitation, a program editing screen for the scenario in which there is an out-of-bounds result for an electrode that is being used to deliver a therapy.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Impedance measurements may be used to determine the electrical integrity of possible stimulation pathways and the electrical integrity of possible electrical sensing pathways. The impedance measurement may be used to classify the electrodes as being a good or valid impedance and/or being an out-of-bounds or invalid impedance. Alternative and/or additional classifications may be used. Invalid impedances may cause a loss in therapy efficacy or increase power consumption as higher output settings may be needed to deliver effective stimulation. Impedance measurements may be scheduled to automatically occur or may be requested by a user, clinician, device rep, or other person. For example, an impedance measurement may be taken when it is determined that stimulation is no longer being delivered or that delivered stimulation has lost its efficacy.

Impedance information may be used to determine whether a therapy should be reprogrammed to avoid using certain electrode(s), or to determine whether the lead(s) should be explanted and another lead implanted. The reprogramming may be performed by a user after being informed that certain electrodes have invalid impedance. In some embodiments, the system may automatically suggest modifications to the programmed therapy (e.g., substituting other electrode(s) for the electrode(s) are determined to have invalid impedance). In some embodiments, the system may automatically lock out electrode(s) that are determined to have invalid impedance(s) from being used in a programmed therapy. For example, the system may prevent a current program from being implemented when the current program is programmed with a therapy that includes an electrode with an invalid impedance, and/or may prevent a user from using an electrode with an invalid impedance in a new or updated program. For example, if it is determined that a first electrode and a second electrode are invalid and that the first electrode is being used in the program, then the system may prevent the first and second electrodes from being used in an updated program.

By way of example and not limitation, some embodiments may use a constant current system to deliver the neurostimulation. With constant current systems, impedances that are too small (e.g., “shorts”) and too large (e.g., “open”) may be simply treated together as impedances that are out of range or out of bounds. For example, impedances below a first threshold may be determined to be too low, and impedances above a second threshold may be determined to be too high. By way of example and not limitation, the first threshold may be 50 Ohms or 100 Ohms. The second threshold may be 2,000 Ohms or 2,500 Ohms. In contrast, voltage systems may attempt to respond to a short by delivering more current over the small impedance. As such, a voltage system may want to distinguish between impedances that are too small and impedances that are too large.

The people who are programming the therapy, receiving a therapy from device or otherwise interacting with the device may not understand or appreciate significance of the impedance measurements. Various embodiments provide an intuitive user interface to inform the user of the impedance status using representations associated with electrode icons that correspond to physical electrodes. Thus, the user may be quickly informed if electrode is valid or invalid.

Various embodiments display impedance results within therapy programming screens. For example, the therapy programming screens may identify the electrodes that have valid impedances and/or the electrodes that have invalid impedances. The actual measurements may be provided in other screens. The display of the impedance results within the therapy programming screens makes it easier for the user to read, interpret, and take actions on the results, as the users do not have to toggle between or among two or more display screens.

The system may provide impedance results with respect to the electrode/lead in therapy programming screens and in impedance results screens. The therapy programming screens (e.g., therapy overview and therapy editing screens) provide the pertinent information about which electrodes have a valid impedance and/or which electrodes have an invalid impedance where the user is determining which of the electrodes should be “active electrodes” used to program the therapy. The active electrodes are those electrodes that are programmed either as an anodic electrode or a cathodic electrode used to deliver the therapeutic energy to the patient. The impedance results screens may provide more detailed impedance information.

Some embodiments provide an “X” or other representation on or adjacent to the electrode(s) on the lead(s) with invalid impedances. The use of the “X” helps solve one of the real-world problems with interpreting impedance results, as most users are not electrical engineers that completely understand what all of those results are telling them numerically and struggle to tie reported results to the actual use/patient use impact decision that they can make. The “X” may be provided only for electrode(s) with invalid impedances that are being used in a programmed therapy. When there is no “X”, the user should be aware that there out of bounds results and they need to take that into consideration if they choose to do any electronic remapping of electrodes. When there is an “X”, the user should attempt to program around the electrode if the user has reported a change in efficacy of the originally programmed therapy. For example, there are no “X” electrodes, then no action needs to be taken. Even if there are electrode(s) with invalid impedances, no X indicates that the invalid electrode is not being used to deliver the therapy. Therefore, no action needs to be taken. If there are some electrodes with invalid impedances and there is an X, then an electrode with invalid impedance is used in therapy. If the patient is still getting effective therapy with the electrode with invalid impedance, then the user may still choose to do nothing. However, if there are some electrodes with invalid impedances and there is an X and the patient is not getting effective therapy, then the user may attempt to modify the electrode configuration of the indicated therapy program target. Some embodiments may provide a system that automatically locks out electrode(s) that are determined to have invalid impedance(s) from being used in a programmed therapy. Thus, a user is unable modify a therapy to use an electrode with an invalid impedance.

By way of example and not limitation, a clinician may request an impedance measurement. If the results indicate that the impedances for all electrodes are within an expected range (e.g., impedance state is valid) and if the patient has not reported any loss of therapy, then no action may be required. The system may be designed to provide an impedance status representation on the user interface (e.g., display screen) for electrodes with valid impedance. Some system embodiments may be designed to provide an impedance status representation only for electrodes with an invalid impedance, such that the absence of the representation indicates that that electrodes have a valid impedance. If the results indicate that the impedances for all electrodes are within an expected range (e.g., impedance state is valid) but the patient indicates that the therapy has become less effective, the multiple impedance measurements may be made to try to identify an intermittent open or short in the electrical pathway. If the open or short is found, then the therapy may be reprogrammed to avoid the electrode with the intermittent impedance issue. If the results indicate that an electrode on one lead is suspect as having an invalid impedance but the patient has not reported any loss of efficacy, then the clinician may determine if that electrode on that lead is used to deliver therapy for any configured therapy program. If that electrode on that lead is not used in any program, then no action needs to be taken. However, if the patient reports a loss of efficacy for the therapy and the electrode suspected to have the invalid impedance is being used within a therapy program, then the clinician may attempt to reprogram the therapy using one or more other electrodes on the lead. If the reprogramming is not effective, then the patient may be a candidate to have the lead replaced.

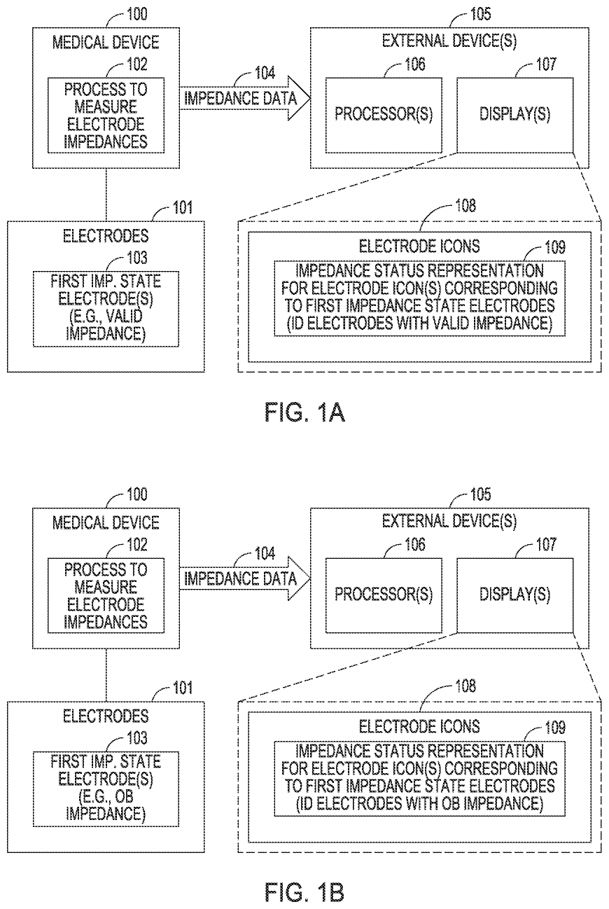

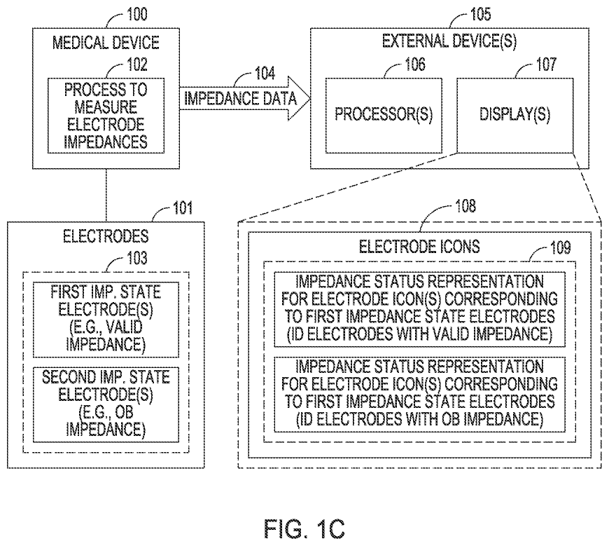

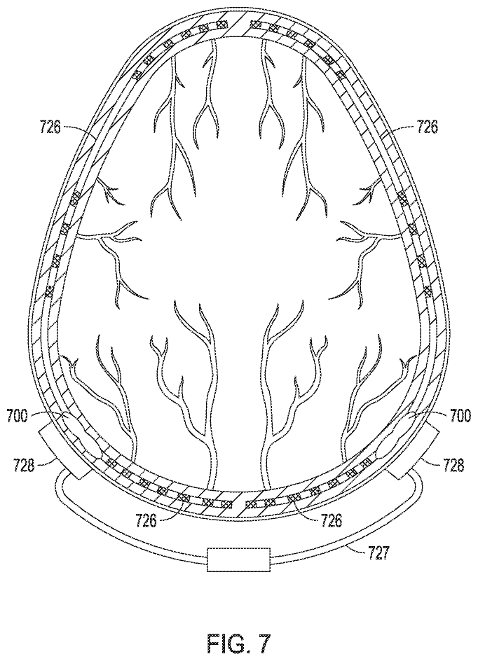

FIGS. 1A-1C illustrate, by way of example and not limitation, various systems for providing electrode impedance status. The illustrated systems include a medical device 100 with a plurality of electrodes 101, which may be on one or more leads. The medical device 100 may be configured to implement a process measure electrode impedances 102. Impedance may be determined by testing pair-wise electrode measurements. For example, in an eight-electrode lead with electrodes E0 through E7, impedance may be measured between all electrode pairs, including the E0-E1 pair, the E0-E2 pair, the E0-E3 pair, . . . E5-E6 pair, E5-E7 pair, and E6-E7 pair. As illustrated in FIG. 7, the system may include four head-located leads including a front left lead, rear left lead, front right lead and rear right lead. For such a system design, the impedance measurements may be performed only with electrode pairs from the same lead. Impedance may be measured using subthreshold pulse amplitude. Preferably, one subthreshold pulse is used for each electrode pair. However, more than one pulse with different pulse characteristics (amplitude, pulse width, frequency) may be used.

The electrodes may be used to deliver an electrical therapy such as a neural stimulation therapy, and/or the electrodes may be used to sense electrical activity. The electrodes may include impedance state electrodes 103, which are at least one electrode associated with impedance measurement(s) such that it can be characterized to have an impedance state. For example, the impedance state electrodes 103 may include valid impedance electrode(s) (see, for example, FIG. 1A), may have invalid impedance electrode(s) or OB (out-of-bounds) impedance electrode(s) (see, for example, FIG. 1B), or may have at least a first impedance state and a second impedance state such as both valid and invalid impedance electrodes (see, for example, FIG. 1C). Some systems may be designed to characterize the electrodes as having other impedance state(s) such as, but not limited to, low impedance, high impedance, marginally low impedance or marginally high impedance.

The medical device 100 may be configured to communicate impedance data 104 to one or more external devices 105. The external device(s) may include a programmer, a phone, a tablet, etc. External devices may be configured to communicate with each other via various wireless and/or wired networks. The external device may include remote servers accessed through the Internet. The processed performed by the external device(s) may be performed solely in a single device or the processes may be distributed across more than one of the external devices. The external device(s) includes at least one processor 106 and at least one display 107. The processor(s) 106 may be configured to analyze the impedance data 106 from the medical device 100 to determine whether any of the electrodes should be characterized as an impedance state electrode 103. The processor 106 may be configured to provide on the display 107 a plurality of electrode icons 108 corresponding to the plurality of electrodes, and provide on the display an impedance status representation 109 for one or more of the plurality of electrode icons corresponding to the one or more impedance state electrodes. The system illustrated in FIG. 1A may be configured to display impedance status representation(s) to positively identify electrode(s) determined to have a valid impedance using the representation(s). The system illustrated in FIG. 1B may be configured to display impedance status representation(s) 109 to positively identify electrode(s) determined to have with an invalid or out-of-bounds impedance using the representation(s). The system illustrated in FIG. 1C may be configured to display impedance status representation(s) 109 to positively identify both electrode(s) determined to have a valid impedance and electrodes(s) determined to have an invalid or out-of-bounds impedance.

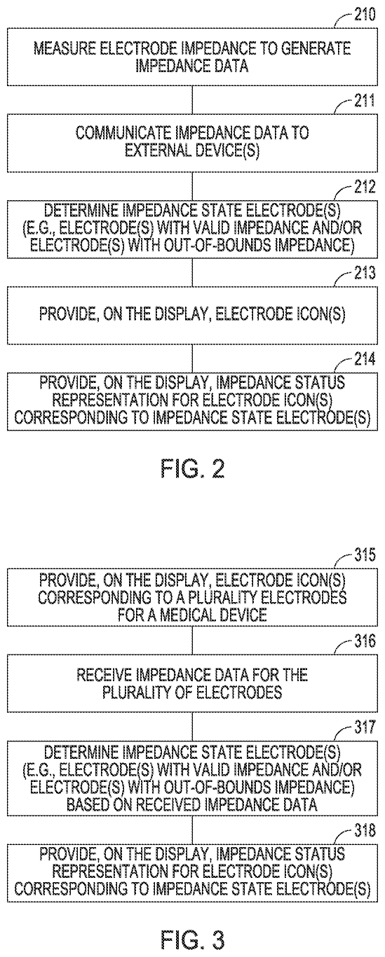

FIG. 2 illustrates, by way of example and not limitation, a method for providing electrode impedance status. The method may be performed using a system that includes a medical device having a plurality of electrodes and at least one external device including a display and a processor. The medical device may be an implantable device or an external device such as but not limited to a wearable device. The illustrated method includes measuring impedance for each of the plurality of electrodes to generate impedance data 210. The impedance may be measured using the medical device. The impedance measurements may be requested by a clinician, a patient, a device rep, or other user according to various embodiments. Some embodiments automatically perform the impedance measurements based on a schedule or based on some other automatically-determined trigger (e.g., sensor-based). The generated impedance data may be communicated from the medical device to the at least one eternal device 211. One or more of the processors may be used to determine, based on the impedance data, one or more impedance state electrodes from the plurality of electrodes that have an impedance state 212. For example, the impedance state electrode(s) may include electrode(s) categorized as having a valid impedance and/or electrode(s) categorized as having an invalid or out-of-bounds impedance. One or more of the processors may provide on the display a plurality of electrode icons corresponding to the plurality of electrodes 213, and may provide on the display an impedance status representation for one or more of the plurality of electrode icons corresponding to the one or more impedance state electrodes 214. By way of example and not limitation, the method may provide on the display representation(s) corresponding to electrode icon(s) to identify electrode(s) that have an invalid impedance and/or identify electrode(s) having a valid impedance.

FIG. 3 illustrates, by way of example and not limitation, a method implemented by a machine (such as a programmer) for providing electrode impedance status. The method may be performed by the machine by implementing instructions on a non-transitory machine-readable medium. The machine-implemented method may include providing on a display a plurality of electrode icons corresponding to a plurality of electrodes for a medical device 315 and receiving impedance data for the plurality of electrodes 316. The impedance data may be received from a medical device that performed impedance measurements. The impedance data may include the raw impedance measurements, or may include processed data (e.g., identifying the electrode(s)) that are categorized into the impedance state. The machine-implemented method may further include determining, based on the impedance data, one or more impedance state electrodes from the plurality of electrodes 317, and providing on the display an impedance status representation for one or more of the plurality of electrode icons corresponding to the one or more impedance state electrodes 318. By way of example and not limitation, the machine-implemented method may determine the impedance state electrodes as electrode(s) having an invalid impedance and/or electrode(s) having a valid impedance.

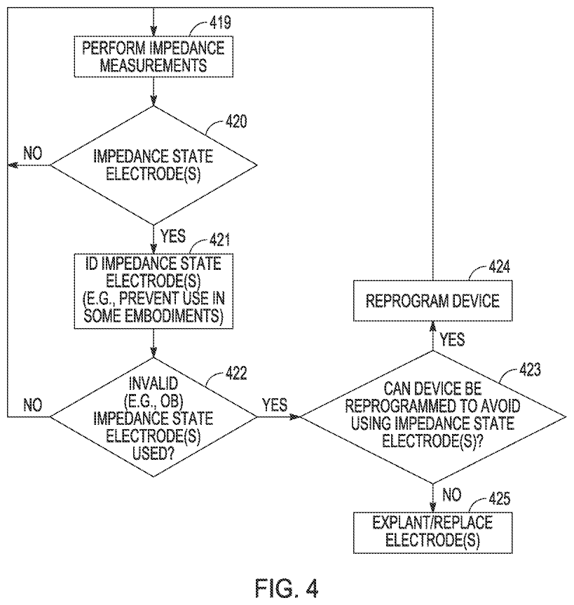

FIG. 4 illustrates, by way of example and not limitation, a method that may be performed using the impedance status. The illustrated method includes performing impedance measurements 419. The impedance measurements may be requested by a clinician, a patient, a device rep, or other user according to various embodiments. Some embodiments automatically perform the impedance measurements based on a schedule or based on some other automatically-determined trigger (e.g., sensor-based). A determination is made, based on the impedance measurements, whether the electrodes include impedance state electrodes (electrodes having an impedance that can be characterized as being an impedance state 420 (e.g., a valid impedance or an invalid impedance). If there are no impedance state electrodes, the process may return to 419 until the next impedance measurement is taken. At 421, the method identifies the impedance state electrodes to a user for a user to determine how to proceed. The method may determine whether any invalid impedance state electrode(s) are being used in one or more of the therapies programmed into the medical device 422. If there are no invalid impedance state electrode(s) used in any of the therapies, the process may return to 419 until the next impedance measurement is taken. If an invalid impedance state electrode(s) is used in at least one of the therapies, then a clinician or device rep may determine whether to attempt to reprogram the therapy to avoid using the invalid impedance state electrodes 423. Reprogramming may be appropriate when the patient indicates that the therapy has lost efficacy. If the device can be successfully reprogramed to deliver an efficacious therapy without the invalid impedance state electrode, then the device is reprogrammed and therapy resumes at 424, and the process returns to 419 until the next impedance measurement is taken. If the device cannot be reprogrammed to deliver an efficacious therapy without the invalid impedance state electrode, then the lead with the invalid impedance state electrode may be explanted and replaced with a different lead 425.

By way of example and not limitation, a medical device system may include implantable medical device(s) and an external device configured for use to communicate with and/or charge the implantable medical device(s). More particularly, the system may include a fully head-located neurostimulator(s) designed for the treatment of chronic head pain. The system may be configured to provide neurostimulation therapy for chronic head pain, including chronic head pain caused by migraine and other headaches, as well as chronic head pain due other etiologies. For example, the system may be used to treat chronic head and/or face pain of multiple etiologies, including migraine headaches; and other primary headaches, including cluster headaches, hemicrania continua headaches, tension type headaches, chronic daily headaches, transformed migraine headaches; further including secondary headaches, such as cervicogenic headaches and other secondary musculoskeletal headaches; including neuropathic head and/or face pain, nociceptive head and/or face pain, and/or sympathetic related head and/or face pain; including greater occipital neuralgia, as well as the other various occipital neuralgias, supraorbital neuralgia, auriculotemporal neuralgia, infraorbital neuralgia, and other trigeminal neuralgias, and other head and face neuralgias.

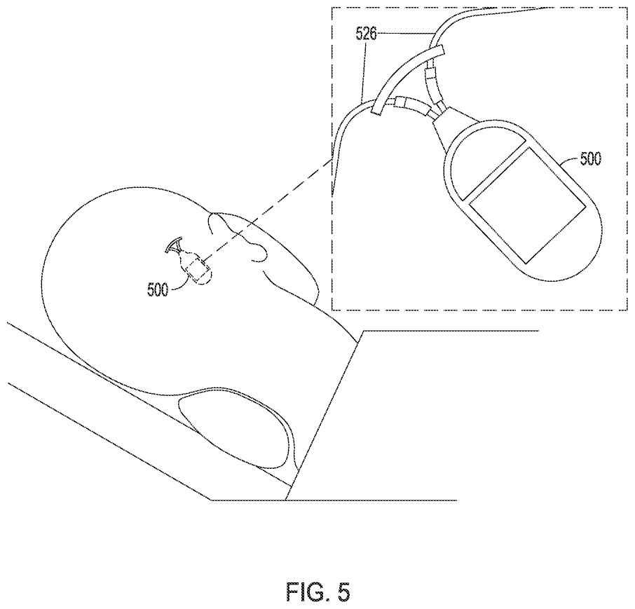

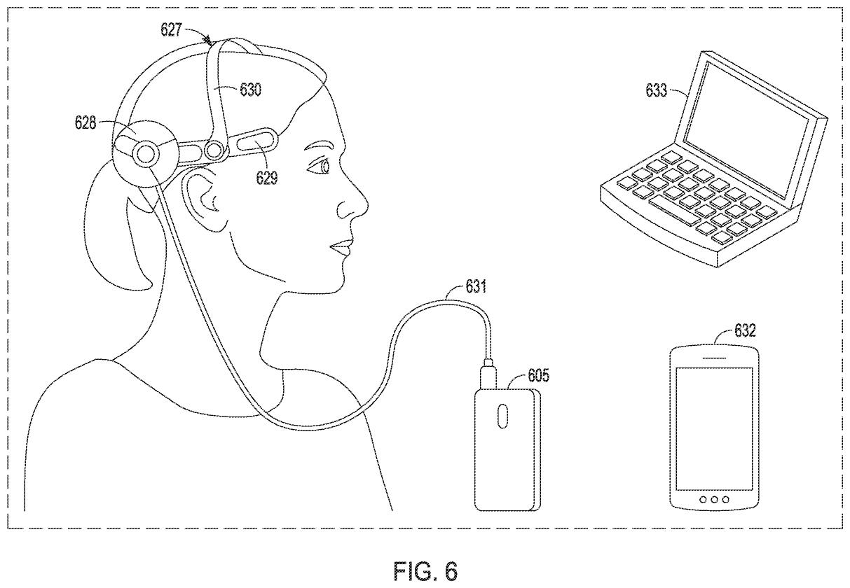

FIG. 5 illustrates an implantable medical device 500 implanted beneath the skin and over a patient's cranium. The device 500 is illustrated as being implanted behind and above the ear. The implantable medical device 500 may include one or more leads 526 that may be subcutaneously tunneled to a desired neural target. Each lead may include one or more electrodes. The number of electrodes and spacing may be such as to provide therapeutic stimulation over any one or any combination of the supraorbital, parietal, and occipital region substantially simultaneously. The implantable medical device 100 may be configured to independently control each electrode to determine whether the electrode will be inactive or configured as a cathode or an anode. One or more electrodes on the lead(s) may be configured to function as an anode, and one or more electrodes on the lead may be configured to function as a cathode. For example, bipolar neuromodulation may be delivered using one or more anodes and one or more cathodes on the lead(s). A clinician may program the electrode configurations to provide a neuromodulation field that captures a desired neural target for the therapy.

FIG. 6 illustrates an external device 605 and headset 627 configured for use to communicate with and/or charge the implantable medical device(s). The headset 627 may include an external coil 628, and the headset 627 may be configured to position the external coil over an implantable medical device. For example, the headset 627 may include an adjustable frame 629 on each side of the head that can rotate about a point on a main headset frame 630, and may be configured to provide additional degrees of motion (e.g., sliding or pivoting motion) with respect to the main headset frame 630. These adjustable frames may be used to position the external coils 628 over the implantable medical devices when the main headset frame 630 is worn. The external device 605 may be electrically connected to the external coil 628 via a cable 631. In some embodiments, the external device 605 may be wirelessly connected to the headset 627. The headset may be configured to wirelessly receive power from the external device and to transfer power from the external coil to the implanted device(s). The external device may communicate, using wireless or wired communication technology, with other external devices such as a phone 632 or programmer 633.

FIG. 7 illustrates, by way of example and not limitation, two implanted devices 700 with leads 726 to cover both sides of the head with one of the devices on the left side of the head and the other on the right side of the head, and a charging/communication headset 727 disposed about the cranium. The headset 727 may include right and left coupling coil enclosures, respectively that contain coils for coupling to the respective coils in the implants. The coil enclosures may interface with a main charger/processor body which may contain processor circuitry and batteries for both charging the internal battery in the implantable medical devices 700 and also communicating with the implanted devices. Thus, in operation, when a patient desires to charge their implanted devices 700, all that is necessary for some embodiments is to place the headset about the cranium with the coils 728 in close proximity to the respective implanted devices 700. In some embodiments, such placement may automatically initiate charging; whereas in other embodiments, the user may initiate charging using an external device. When the headset 727 is worn by a patient, the headset coils (transmit coils) 728 are placed in proximity to the corresponding receive coil in each respective body-implanted implantable medical device 700. The headset 727 may include telemetry circuitry, a controller, a battery, and a Bluetooth wireless interface. The headset 727 may also communicate with a personal device such as a smartphone or tablet (e.g., via the Bluetooth interface), for monitoring and/or programming operation of the two implantable medical devices.

The implantable medical device 700 may include a rechargeable battery, an antenna (e.g., coil), and at least one Application Specific Integrated Circuit (ASIC), along with the necessary internal wire connections amongst these related components, as well as to the incoming lead internal wires. These individual components may be encased in a can made of a medical grade metal, which may be encased by plastic cover. The battery may be connected to the ASIC(s) via a connection that is flexible. The overall enclosure for the battery, antenna and ASIC(s) may have a very low flat profile with two lobes, one lobe for housing the ASIC(s) and one lobe for housing the battery. The antenna may be housed in either of the lobes or in both lobes. The use of the two lobes and the flexible connection between the ASIC(s) and the battery allows the implanted device to conform to the shape of the human cranium when subcutaneously implanted without securing such to any underlying structure with an external fixator.

The ASIC(s) and lead may be configured to independently drive each of the electrodes using a neuromodulation signal in accordance with a predetermined program. The programmed stimulation may be defined using parameters such as one or more pulse amplitudes, one or more pulse widths and one or more pulse frequencies. Other parameters may be used for other defined waveforms, which may but does not necessarily use rectilinear pulse shapes. Once the program is loaded and initiated, a state machine may execute the particular programs to provide the necessary therapeutic stimulation. The ASIC(s) may have memory and be configured for communication and for charge control when charging a battery. Each of the set of wires interface with the ASIC(s) such that the ASIC(s) individually controls each of the wires in the particular bundle of wires. Thus, each electrode may be individually controlled. Each electrode may be individually turned off (inactive), or as noted above, each electrode may be individually activated and designated as an anode or a cathode. During a charging operation, the implanted device is interfaced with an external charging unit via the antenna (e.g., coil) which is coupled to a similar antenna (e.g., coil) in the external charging unit. Power management involves controlling the amount of charge delivered to the battery, the charging rate thereof and protecting the battery from being overcharged.

The ASIC(s) may be capable of communicating with an external unit, typically part of the external charging unit, to exchange information. Thus, configuration information can be downloaded to the ASIC and status information can be retrieved. A headset may be provided for such external charging/communication operation.

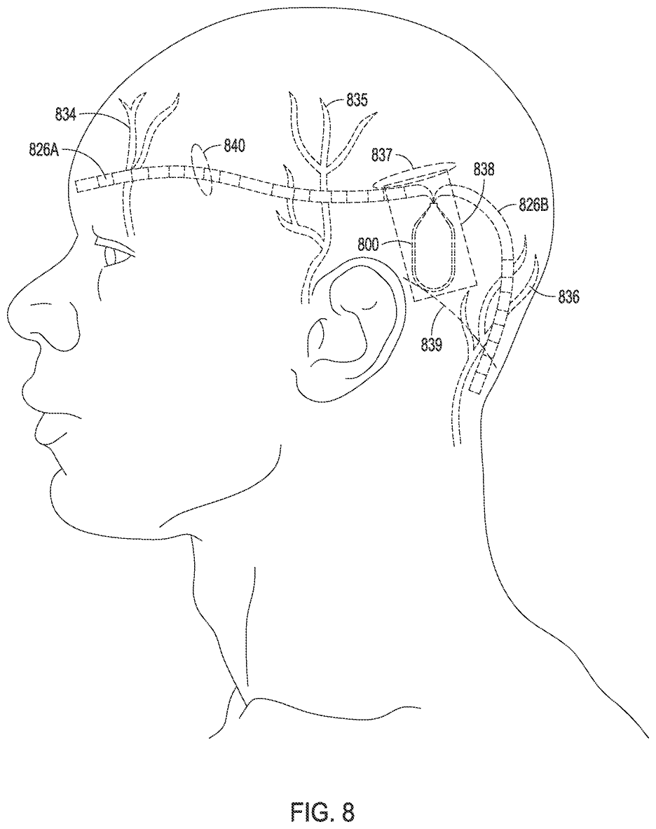

FIG. 8 illustrates, by way of example and not limitation, a side view of fully head-located neurostimulation system. Also illustrated are the supraorbital nerve 834, the auriculotemporal nerve 835 and the occipital nerve 836. The illustrated system includes a medical device (e.g., pulse generator) 800 and both a first lead 826A and a second lead 826B attached to the medical device 800. The medical device 800 and leads 826A and 826B are configured for use in stimulating the supraorbital nerve 834 toward the front of the head and the occipital nerve 836 toward the back of the head.

The patient may have had a period of trial neurostimulation, which is standard in traditional neurostimulator evaluations but is optional here. The actual permanent implant may occur in a standard operating suite with appropriate sterile precautions. By way of example and not limitation, the patent may be prepped and draped. The patient may be administered prophylactic antibiotics, local anesthetic, and sedation. The patient may be placed in a supine position with a head of the bed elevated to approximately thirty degrees. The patient's head may be turned to better access the intended implant location. While the implantable medical device may be positioned subcutaneously anywhere, it may be positioned above and behind the ear in this illustrated embodiment. Thus, a first incision 837 of sufficient length (approximately 4-6 cm) is made to a depth sufficient to reach the subcutaneous layer. A pocket 838 to accept the medical device 800 is fashioned by standard dissection techniques. The pocket 838 may be directed below the incision. The pocket 838 may be angled depending on the desired orientation of the medical device. For example, the pocket 838 may be angled posteriorly, as illustrated. The pocket 838 may be 10-20% larger than the medical device 800 to allow for a comfortable fit and no undue tension on the overlying skin and/or incision. The first incision 837 may be made and the pocket 838 formed so that the implantable medical device abuts against the nuchal ridge 839 when fully inserted into the pocket 838. The first incision 837 should not interfere with the implanted medical device 800. The present subject matter may use template(s) to help make the incision in a desired location.

A second incision 840 may be made to the subcutaneous layer at a point above and anterior to the pinna of the ear in the temple region to assist with subcutaneously routing the first lead 826A. The first lead 826A may be passed from the medical device 800 in the pocket 838 to the second incision 840, and then passed from the second incision 840 to its final subcutaneous position over supraorbital nerves 834. The second lead 826B may be passed from the medical device 800 in the pocket 838 back toward the occipital nerve 836. The medical device 800 may be inserted into the pocket 838 either before or after the leads 826A and/or 826B are tunneled to their final subcutaneous position to deliver therapy.

Tubular introducer(s) with a plastic-peel away shell may be used to assist with lead placement. However, other techniques may be used to subcutaneously tunnel the leads to their final placement to deliver the neurostimulation therapy. Following the entire placement of the complete system, including the medical device and both leads and suturing, the medical device may be powered-up and its circuits checked. Upon recovery from anesthesia the system may be turned on for the patient with a portable programmer and the multiple parameters for the system may be programmed to provide a desired therapy for the patient.

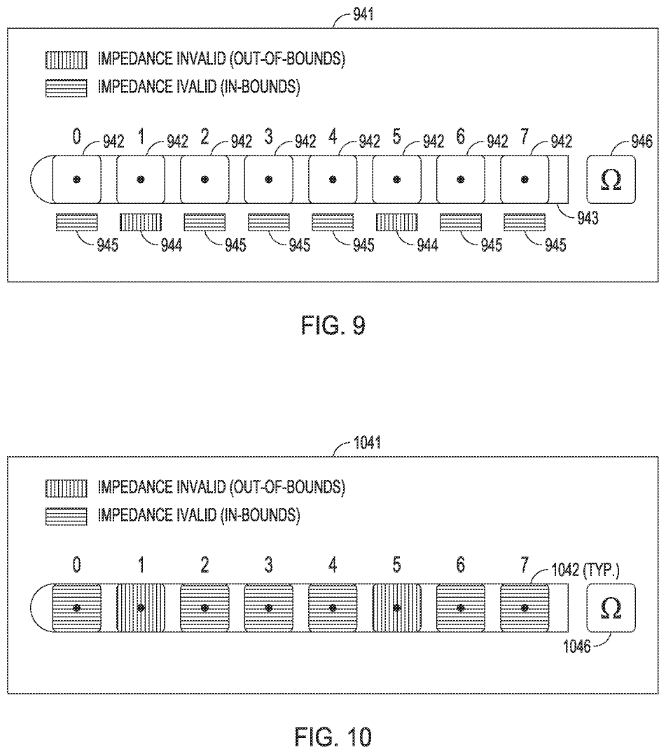

FIG. 9 illustrates, by way of example, and not limitation, a display with electrode icons and some examples of impedance status representations for the electrode icons. The display 941 may be presented on a user interface of an external device. The display 941 includes a plurality of electrode icons 942 corresponding to a plurality of electrodes of a medical device. The electrodes may correspond to the electrodes on a medical device lead. The display may include a corresponding lead representation 943. The display may include an impedance status representation for one or more of the plurality of electrode icons corresponding to the one or more impedance state electrodes. The impedance status representation may be for impedance state electrode(s) that have an out-of-bounds impedance 944, and/or the impedance status representation may be for impedance state electrode(s) that have a valid impedance 945. Although both are displayed, it is expressly noted that the system may be configured to display only one of the valid representation or invalid representation. Also, the impedance status representations 944 945 are illustrated with a pattern. A color may be used to identify the one or or more impedance status for the electrodes.

Notably, the display provides impedance status information for the electrodes to the user without displaying the actual measurements. The display may include a representation of the lead with the plurality of electrode icons. Each of the electrode icons may be labeled with a corresponding electrode number. In the illustrated embodiment, the impedance status representation includes a line segment under the one or more of the plurality of icons corresponding to the one or more impedance state electrodes. The line segments may be above the electrodes or otherwise near the electrodes such that the easily identify that electrode that corresponds to an impedance status representation. FIG. 9 also illustrates a control 946 which may be labeled with the Greek letter Omega for “Ohms.” Selection of the control may trigger the medical device to perform an impedance test.

FIG. 10 illustrates, by way of example, and not limitation, a display with electrode icons and other examples of impedance status representations for the electrode icons. The display 1041 in FIG. 10 is similar to the display 941 in FIG. 9. The display 1041 may be presented on a user interface of an external device. Rather than being a representation, such as a line segment, adjacent to the electrode, FIG. 10 illustrates an example in which the electrodes icons 1042 are changed to illustrate which electrode(s) have an invalid impedance and/or which electrode(s) have a valid impedance). For example, different patterns, outlines or colors may be used to provide the impedance status representation for the icons. FIG. 10 also illustrates a control 1046 which may be labeled with the Greek letter Omega for “Ohms.” Selection of the control may trigger the medical device to perform an impedance test.

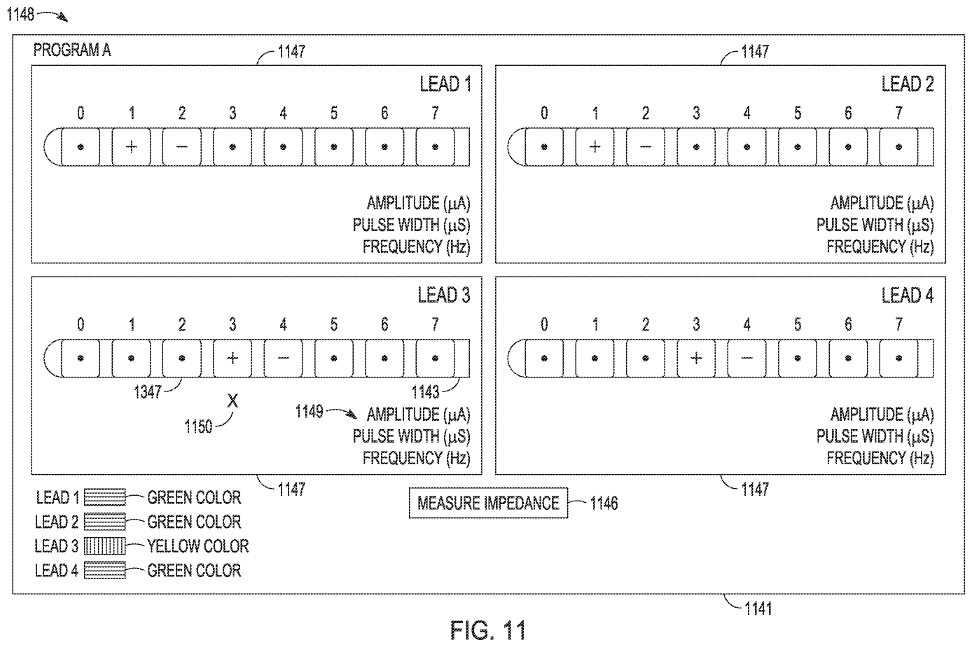

FIG. 11 illustrates, by way of example and not limitation, a therapy programming display screen configured for use to program an electrical therapy including selecting active ones of the plurality of electrodes for use to deliver the electrical therapy, where the therapy programming display screen includes the impedance status representation. The illustrated programming screen 1141 includes four leads (e.g., lead cards 1147) for Program A. The collection of the lead cards 1147 in a program may be referred to as a therapy card 1148). The four lead cards may correspond to the four leads 726 illustrated in FIG. 7 Each illustrated lead representation 1143 within a lead card 1147 includes corresponding electrode representation 1142. The electrode representations may be numbered (e.g., an eight-electrode lead may have the electrodes numbered 0-7 or numbered 1-8). Each lead may include a programmable electrical waveform configuration 1149 (e.g., pulse amplitude, pulse width and pulse frequency). A clinician, device rep or other user may use the programming display screen to determine which of the available electrodes are to be the active electrodes for the therapy and which electrodes are inactive (electrode icons with “·” symbol or without a negative sign “−” or positive sign “+”. The user may select which of the active electrodes are cathodic electrode illustrated with the negative sign “−” and one anodic electrode illustrated with the positive sign “+”. The illustrated programming display screen includes a “Measure Impedance” control 1146. Selection of this control trigger may trigger the medical device to perform an impedance test. The system may determine from the results of the impedance test that Electrode 3 in Lead 3 has an invalid impedance state (i.e., Electrode 3 is an invalid impedance state electrode). As Electrode 3 in Lead 3 is currently configured as an active electrode in Program A, the system may identify that electrode with an “X” 1150, indicating that the clinician, device rep or other use should determine whether the patient has lost efficacy and/or whether Program 1 can be reprogrammed to select other active electrodes for Lead 3 in place of Electrode 3. Each lead may include a representation (e.g., color, pattern and/or words indicating the impedance status representation for that lead). For example, the color green may indicate that all active electrodes on the lead used in the program have valid impedances, whereas the color yellow may indicate that the program is using at least one electrode on that lead that has an invalid impedance. The different colors are illustrated by different patterns in FIG. 11.

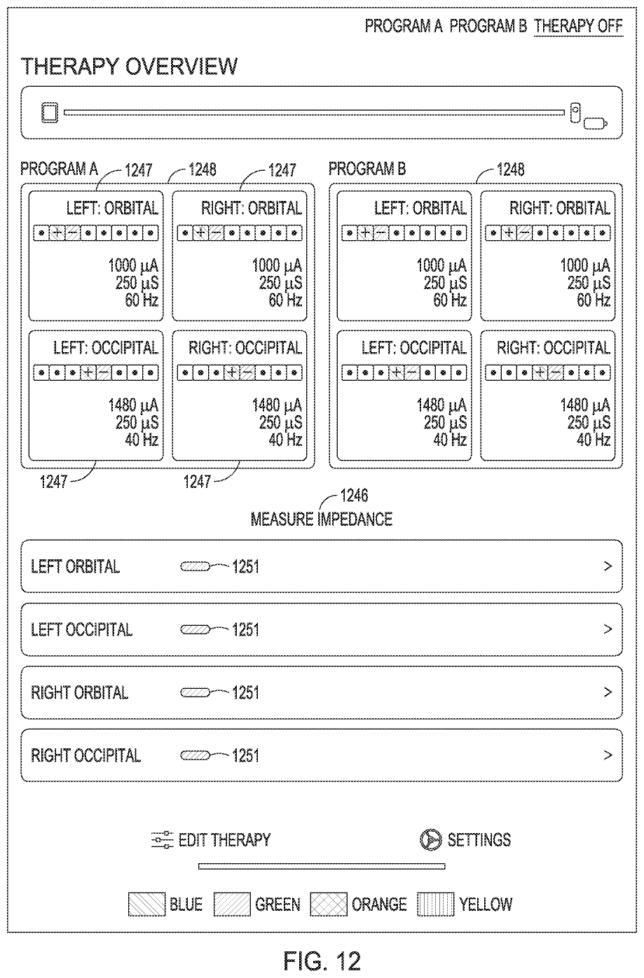

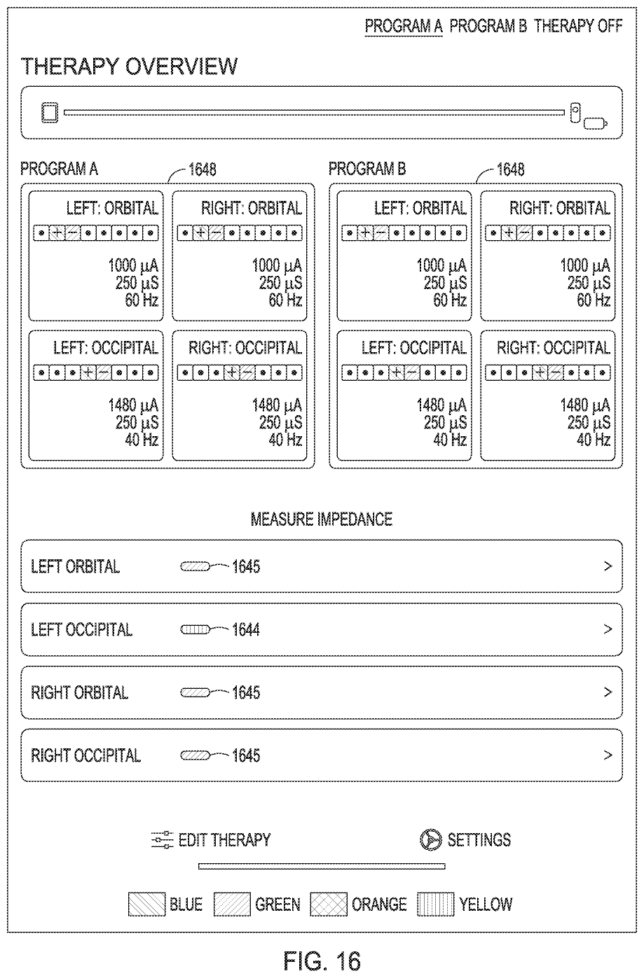

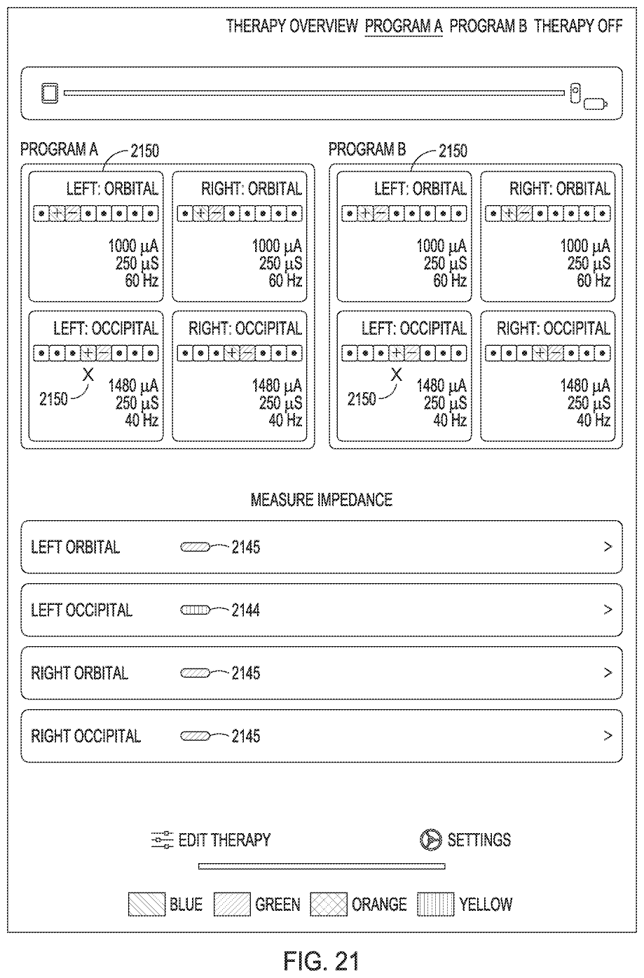

FIGS. 12-15 illustrate a user interface after an impedance measurement when all reported results are within bounds, FIGS. 16-20 illustrate the user interface after an impedance measurement when there is an out-of-bounds result for an electrode that is not being used to deliver a therapy, and FIGS. 21-25 illustrate the user interface after an impedance measurement when there is an out-of-bounds result for an electrode that is being used to deliver a therapy as identified by the “X” representation.

FIG. 12 illustrates, by way of example and not limitation, a therapy overview screen with more than one therapy program and more than one lead for each program. FIG. 12 illustrates a scenario where all results of the impedance measurements are valid (e.g., within an acceptable range). The therapy overview screen may include a representation (e.g., which may be referred as a “therapy card” 1248 for each programmed therapy (e.g., Program A and Program B). The activated therapy card may have a thicker border. Each therapy card may include representations (e.g., which may be referred to as a “lead card” 1247) for each lead in the system. In the illustrated embodiment representing the system illustrated in FIGS. 5-8, each program may be divided into four Neurostimulator Lead Cards: “Left: Orbital,” Left: Occipital,” “Right: Orbital,” and “Right: Occipital”. Each lead card 1247 may display a title, an illustration of a neurostimulator lead active electrodes and the polarity of the electrodes, and waveform parameters such as a numerical pulse amplitude in μA, a numerical pulse width in μs, and a numerical frequency in Hz.

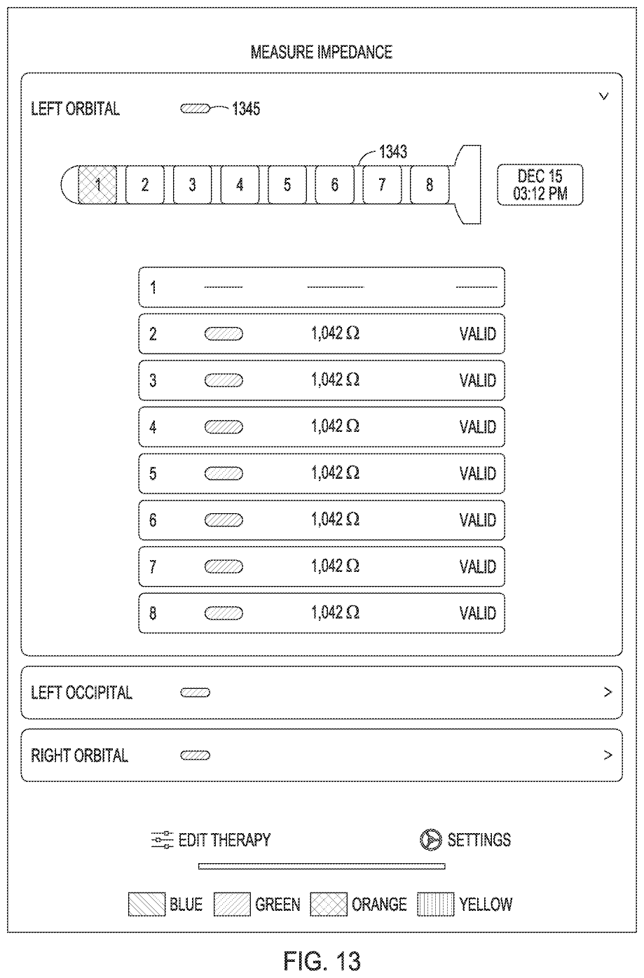

The illustrated therapy overview screen may include clickable text “MEASURE IMPEDANCE” 1246 which will trigger the medical device to perform impedance measurements for all electrode pairs in each lead. An impedance state status for each lead may be displayed near the therapy cards. In FIG. 12, each of the leads has a lead impedance representation (e.g., green) indicating that all active electrodes in that lead have a valid impedance. If the first lead, entitled “Left Orbital,” is selected, the system may proceed to the display screen illustrated in FIG. 13.

FIG. 13 illustrates, by way of example and not limitation, electrode impedance detail for one of the leads. This screen may be presented if the user selected the first listed lead, “Left Orbital” lead, in FIG. 12. FIG. 13 continues to illustrate a scenario where all results of the impedance measurements are valid (e.g., within an acceptable range). The illustrated left orbital lead 1343 has an impedance status representation 1345 (e.g., green) indicating that all electrodes (both active and inactive) in that lead have a valid impedance. FIG. 13 illustrates the eight electrodes on the lead, along with a date and time when the impedance measurements were taken. Electrode 1 has been selected, indicating that the detail provided in this screen is for the impedance measurements between electrode pairs that include electrode 1. FIG. 13 illustrates a table that includes a column listing each electrode, an impedance status representation associated with each electrode (e.g., green bar), the measured impedance value, and text (e.g., “valid”) indicating the impedance status for that impedance measurement. Values are provided for each tested electrode pair that includes the selected electrode 1. The actual impedance measurements for every electrode pair on the selected lead may be presented to the user through this screen.

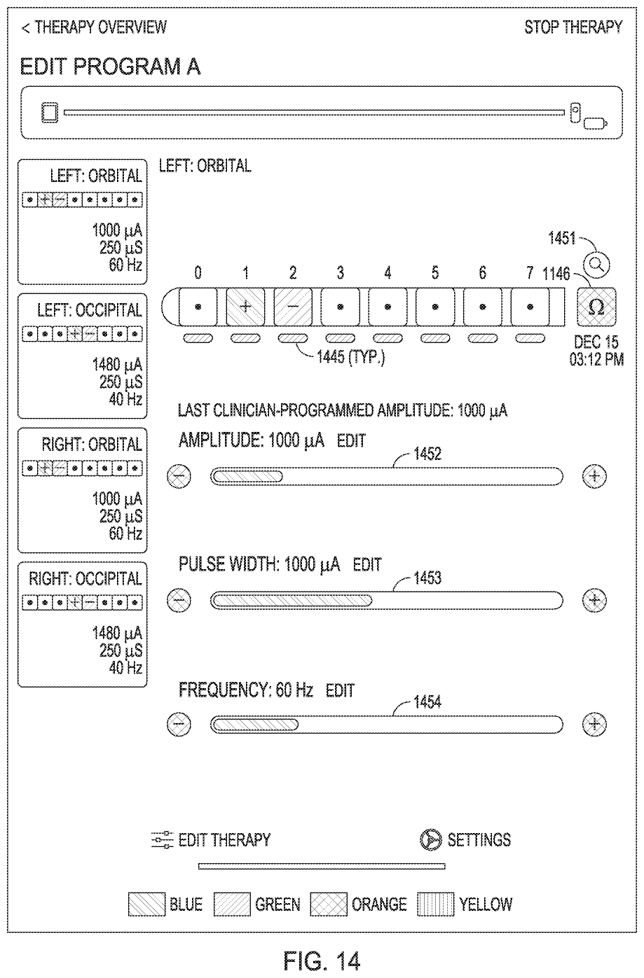

FIG. 14 illustrates, by way of example and not limitation, a programming editing screen. By way of example and not limitation, this screen may be reached by selecting Edit Program from the therapy overview screen in FIG. 12. FIG. 14 continues to illustrate a scenario where all results of the impedance measurements are valid (e.g., within an acceptable range). Program A is illustrated as being edited. The four leads associated with Program A are provided for user selection. In the illustrated figure, the user has selected the Left Orbital Lead. The representation of the lead and the electrodes on the lead correspond to the selected Left Orbital Lead. Each Electrode is labeled (1-8), and each electrode can have polarity assigned or deactivated. According to some embodiments, rules are enforced such that each lead has at least one electrode assigned to negative polarity and at least one electrode assigned to positive polarity, or have all electrodes deactivated. The date and time may be shown near the Patient Lead. The programming editing screen allows the user to change the pulse amplitude via an amplitude bar 1452, change pulse width via the pulse width bar 1453, and change the pulse frequency via the frequency bar 1454. Each bar allows the user to incrementally increase or decrease the value with the + and − icons on the opposing sides of bar. The values may be entered via text by selected the edit next to the corresponding waveform parameter. The representation of the lead includes invalid impedance representations 1445 (e.g., green underscore) next to electrodes with valid impedance measurement. The display includes a control 1451 (a clickable magnifying glass icon) for causing the system to display the impedance details screen, and another control 1146 (Omega) for triggering the medical device to perform an impedance measurement.

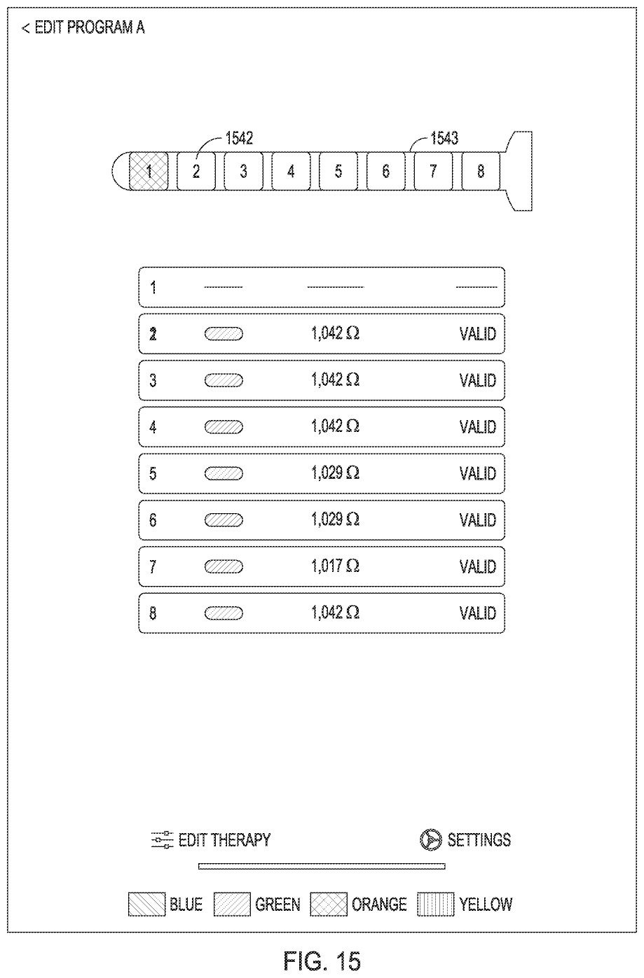

FIG. 15 illustrates, by way of example and not limitation, an electrode impedance detail screen that may be entered by selecting the magnifying glass control 1451 in FIG. 14. FIG. 15 continues to illustrate a scenario where all results of the impedance measurements are valid (e.g., within an acceptable range). Similar to FIG. 13, the display screen in FIG. 15 uses a lead representation 1543 and electrode icons 1542 to illustrate the eight electrodes on the lead, along with a date and time when the impedance measurements were taken. Electrode 1 has been selected, indicating that the detail provided in this screen is for the impedance measurements between electrode pairs that include electrode 1. FIG. 15 illustrates a table that includes a column listing each electrode, an impedance status representation associated with each electrode (e.g., green bar), the measured impedance value, and text (e.g., “valid”) indicating the impedance status for that impedance measurement. Values are provided for each tested electrode pair that includes the selected electrode 1. These values are provided next to the other electrode in the electrode pair that includes the selected electrode 1. The actual impedance measurements for every electrode pair on the selected lead may be presented to the user through this screen, by selecting each electrode on the lead and then viewing impedance values for every electrode paired with the selected electrode.

FIG. 16 illustrates, by way of example and not limitation, a therapy overview screen for a scenario in which there is an out-of-bounds result for an electrode that is not being used to deliver a therapy. As the electrode(s) with the invalid impedance is (are) not being used to deliver the therapy, the Program A therapy card and the Program B therapy card are similar to the therapy cards illustrated in FIG. 12. However, the left occipital lead has an impedance status representation 1644 (e.g., yellow) indicating that at least some electrodes in that lead have an invalid impedance. The other leads have an impedance status representation 1645 (e.g., green) that the electrodes on the lead have a valid impedance. This notifies the user that there is at least one electrode with an invalid impedance on the left occipital lead. However, as the electrode is not being used to deliver the therapy, there is no “X” on the therapy cards for either Program A or Program B.

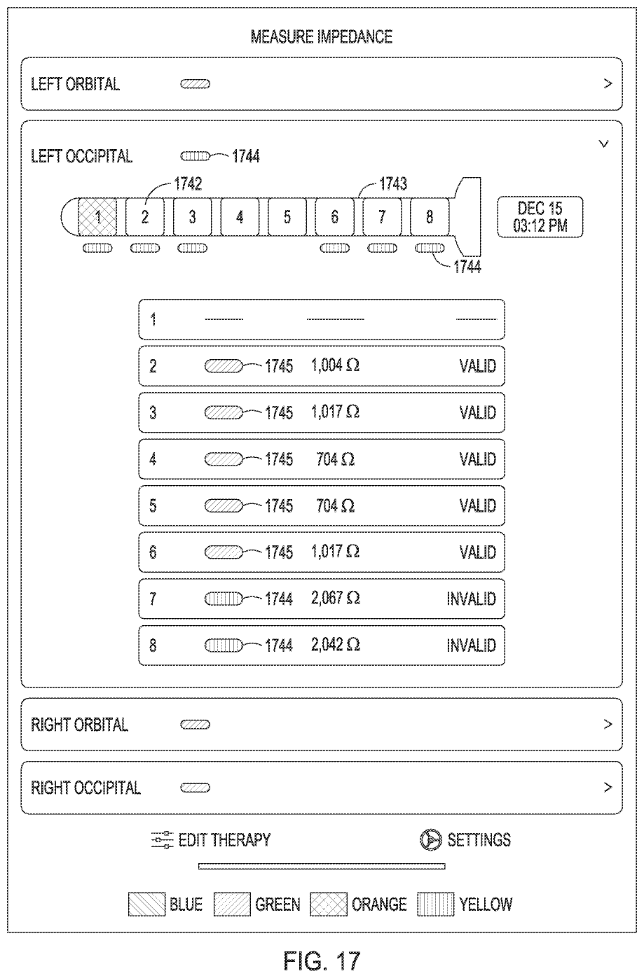

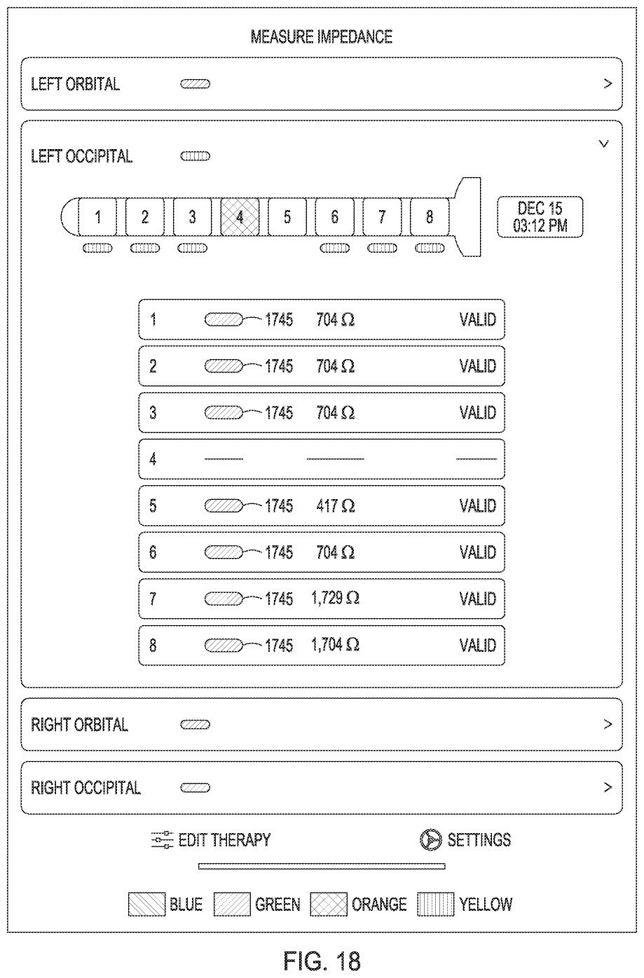

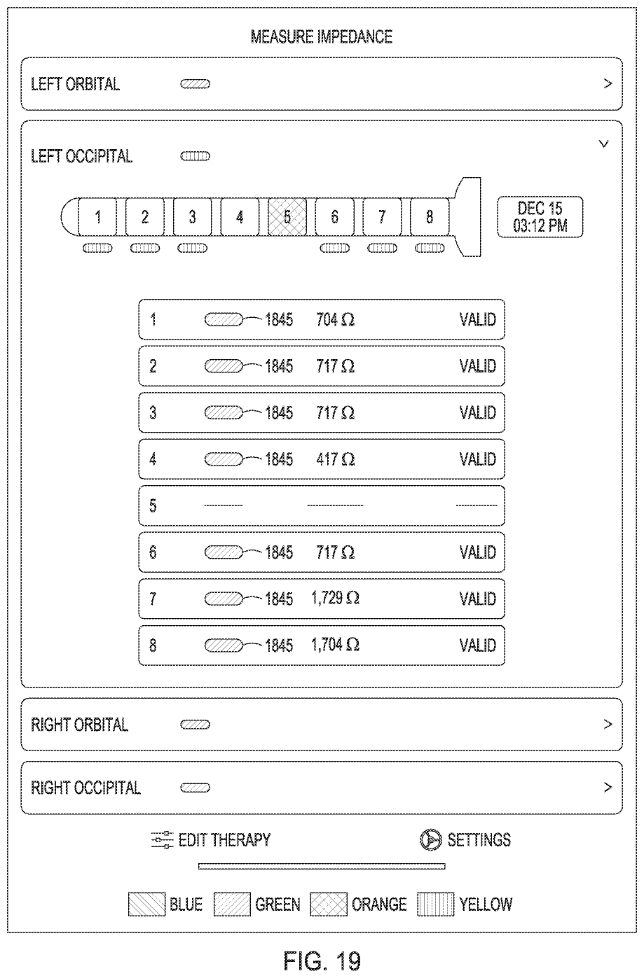

FIG. 17 illustrates, by way of example and not limitation, an electrode impedance detail screen for the Left Occipital lead illustrated in FIG. 16. FIG. 17 continues to illustrate a scenario in which there is an out-of-bounds result for an electrode that is not being used to deliver a therapy. Thus, the user may choose to not address the invalid impedance as it is not causing a problem for the therapy. The left occipital lead has an impedance status representation 1744 (e.g., yellow) indicating that at least some electrodes in that lead have an invalid impedance. FIG. 17 includes a lead representation 1743 and electrode icons 1742 to illustrate the eight electrodes on the lead, along with a date and time when the impedance measurements were taken. Electrodes 1, 2, 3 6, 7 and 8 on the lead have corresponding yellow underscore icons 1744, indicating that each of these electrodes is in an electrode pair that has an invalid impedance. Electrode 1 has been selected, indicating that the detail provided in this screen is for the impedance measurements between electrode pairs that include electrode 1. FIG. 17 illustrates a table that includes a column listing each electrode, an impedance status representation associated with each electrode (e.g., green bar for valid impedances 1745 and yellow bar 1744 for invalid impedances), the measured impedance value, and text (e.g., “Valid” or “Invalid”) indicating the impedance status for that impedance measurement. Values are provided for each tested electrode pair that includes the selected electrode 1. The actual impedance measurements for every electrode pair on the selected lead may be presented to the user through this screen. Thus, the table indicates that, for selected electrode 1, the E1-E7 electrode pair and the E1-E8 electrode pair have invalid impedances. However, it is noted that electrodes 4 and 5 are active for the left occipital lead in the programs (see Therapy Cards in FIG. 16). And as illustrated by the lack of a yellow underscore icon for electrodes 4 and 5 in the lead representation 1743, these electrodes are not associated with an invalid or out-of-bounds impedance. FIG. 18 illustrates, by way of example and not limitation, an electrode impedance detail screen for electrode 4. The therapy detail shows that all electrode pairs involving electrode 4 has valid impedances. FIG. 19 illustrates, by way of example and not limitation, an electrode impedance detail screen for electrode 5. The therapy detail shows that all electrode pairs involving electrode 5 has valid impedances.

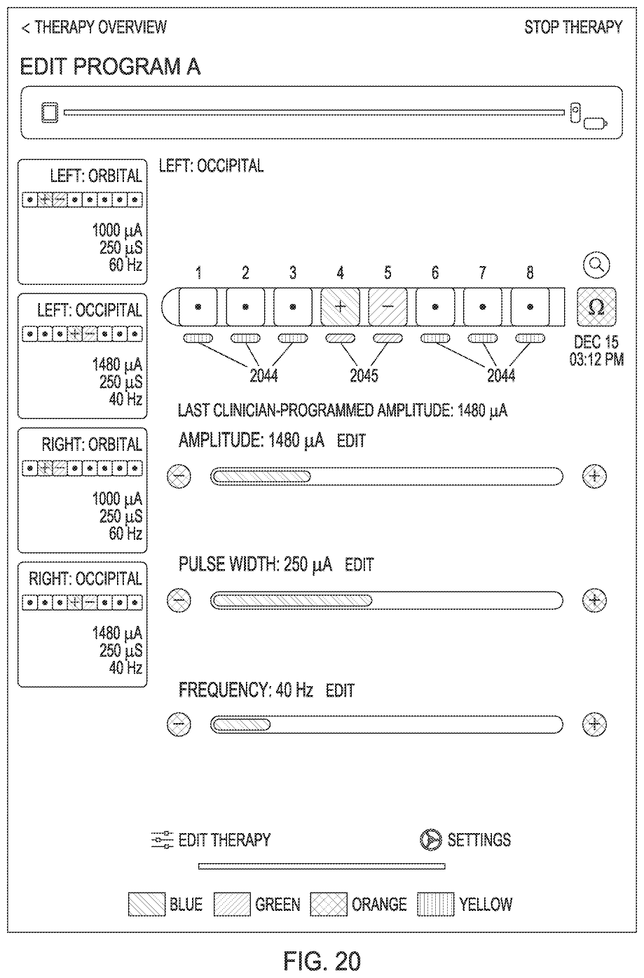

FIG. 20 illustrates, by way of example and not limitation, a program editing screen for the scenario in which there is an out-of-bounds result for an electrode that is not being used to deliver a therapy. The representation of the left occipital lead indicates that electrodes 4 and 5 are active, with electrode 4 configured as an anodic electrode (“+”) and electrode 5 configured as a cathodic electrode (“−”). Both of these active electrodes have a corresponding “valid” underscore impedance status representation 2045 (e.g., green underscore). The inactive electrodes 1, 2, 3, 6, 7 and 8 all have “invalid” underscore impedance representations 2044 (e.g., yellow underscore). However, as none of electrodes 1, 2, 3, 6, 7 and 8 are used in the program therapy, the four lead cards do not include an X next to any of the electrodes with an invalid impedance.

FIG. 21 illustrates, by way of example and not limitation, a therapy overview screen for a scenario in which there is an out-of-bounds result for an electrode that is being used to deliver a therapy as identified by the “X” representation in the left occipital lead card in both of the Program A therapy Card and Program B therapy Card. Similar to FIG. 16, the Left Occipital lead includes an invalid impedance status representation 2144 (e.g., yellow color), whereas the other leads include a valid impedance status representation 2145 (e.g., green color). This notifies the user that there is at least one electrode with an invalid impedance on that lead. However, unlike FIG. 16, the electrode is being used to deliver the therapy as indicated by the “X” 2150 on the program therapy cards 2148.

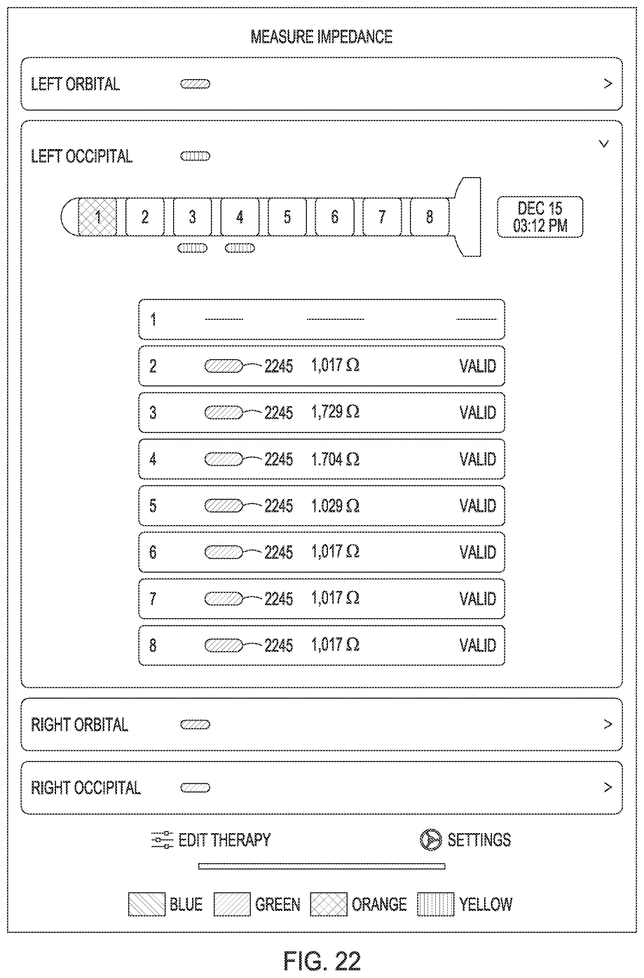

FIG. 22 illustrates, by way of example and not limitation, an electrode impedance detail screen for the Left Occipital lead illustrated in FIG. 21. FIG. 22 continues to illustrate a scenario in which there is an out-of-bounds result for an electrode that is being used to deliver a therapy. Electrode 1 is selected on the representation of the left occipital lead. The therapy detail shows, via a “valid” underscore impedance representation 2245 (e.g., a green underscore), that all electrode pairs involving selected electrode 1 have valid impedances. It is noted that only electrodes 3 and 4 have “invalid” underscore impedance status representations (e.g., yellow underscore). However, the electrode pairs 1-3 and 1-4 still have valid impedance measurements. The other electrodes do not show that they have an invalid impedance, and thus do not have an underscore.

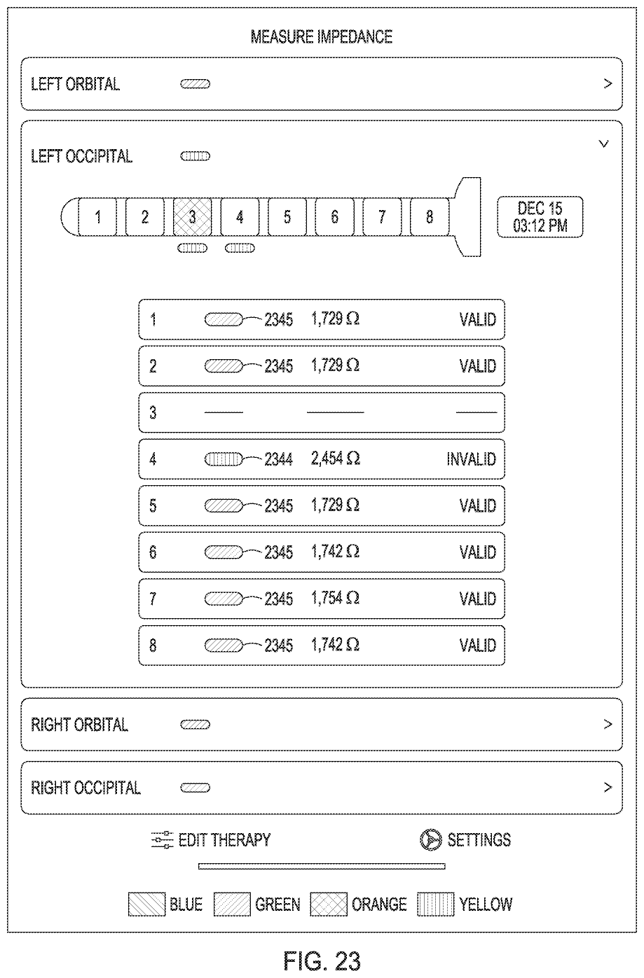

FIG. 23 illustrates, by way of example and not limitation, an electrode impedance detail screen for the Left Occipital lead illustrated in FIG. 21. FIG. 23 continues to illustrate a scenario in which there is an out-of-bounds result for an electrode that is being used to deliver a therapy. Electrode 3 is selected on the representation of the left occipital lead. The therapy detail shows, via an “invalid” underscore impedance representation 2344, that electrode pair E3-E4 has an invalid impedance 2344 of 2,454 Ohms, and shows, via a “valid” underscore impedance representation 2345, all other electrode pairs involving selected electrode 3 has valid impedances.

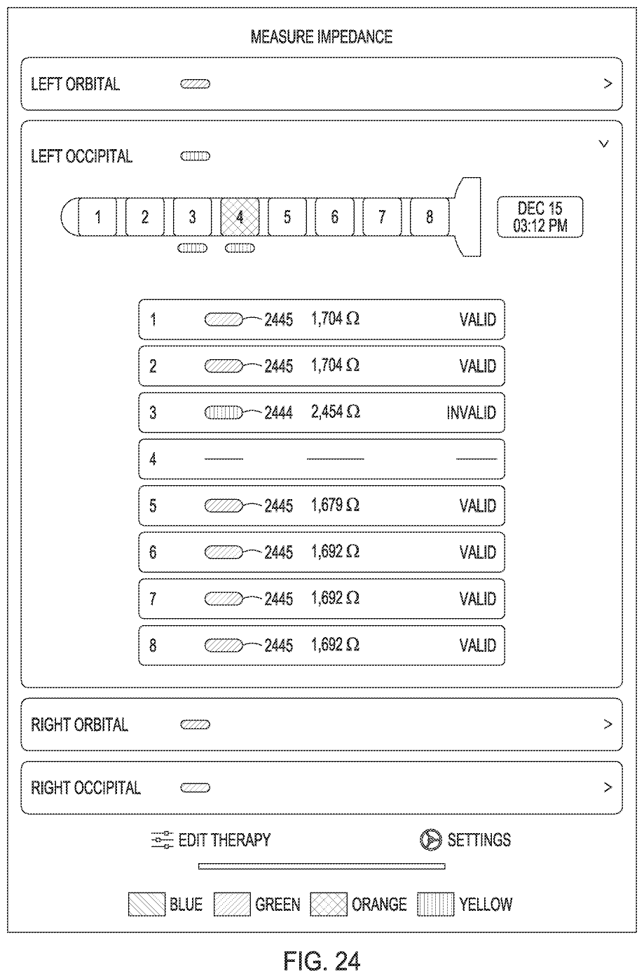

FIG. 24 illustrates, by way of example and not limitation, an electrode impedance detail screen for the Left Occipital lead illustrated in FIG. 21. FIG. 24 continues to illustrate a scenario in which there is an out-of-bounds result for an electrode that is being used to deliver a therapy. Electrode 4 is selected on the representation of the left occipital lead. The therapy detail shows, via an “invalid” underscore impedance representation 2444, that electrode pair E3-E4 has an invalid impedance 2444 of 2,454 Ohms, and shows, via a “valid” underscore impedance representation 2445, all other electrode pairs involving selected electrode 4 has valid impedances.

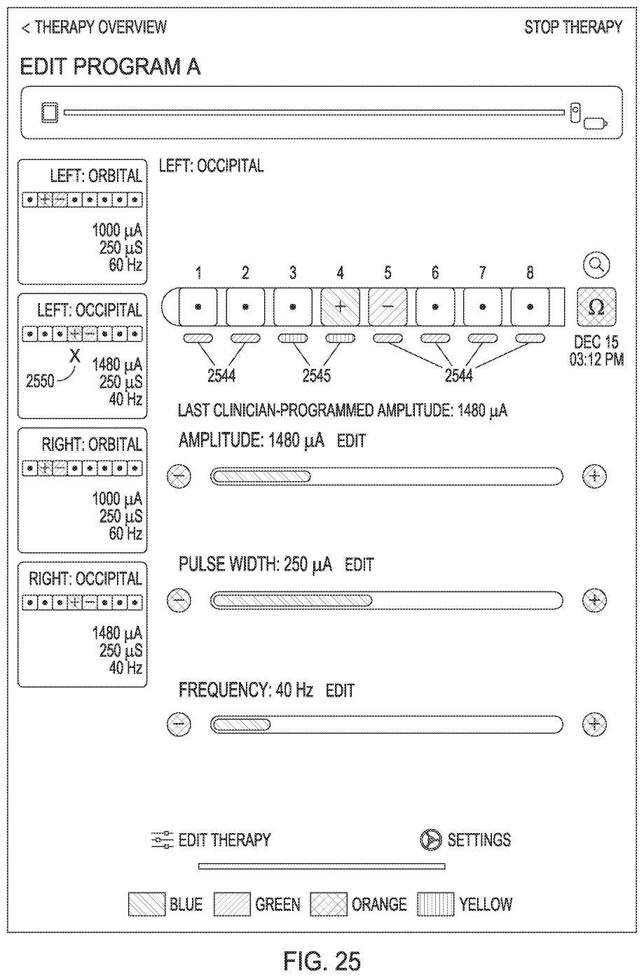

FIG. 25 illustrates, by way of example and not limitation, a program editing screen for the scenario in which there is an out-of-bounds result for an electrode that is being used to deliver a therapy. The representation of the left occipital lead indicates that electrodes 4 and 5 are active, with electrode 4 configured as an anodic electrode (“+”) and electrode 5 configured as a cathodic electrode (“−”). Active electrode 4 has a corresponding “invalid” underscore impedance status representation (e.g., yellow underscore) and active electrode 5 has a corresponding “valid” underscore impedance representation (e.g., green underscore). The inactive electrodes 1, 2, 6, 7 and 8 all have “valid” underscore impedance representations 2545 (e.g., green underscore), and inactive electrode 3 has an “invalid” underscore impedance representation 2544 (e.g., yellow underscore). However, as active electrode 4 is used in the therapy and has an invalid impedance, the left occipital lead card includes an “X” 2550 next to electrode 4.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A system, comprising:
a medical device having a plurality of electrodes, the medical device being configured to measure impedance for each of the plurality of electrodes and generate impedance data; and
at least one external device, the at least one eternal device including a display and a processor, wherein the medical device is configured to communicate the generated impedance data to the at least one eternal device,
wherein the processor is configured to provide on the display a plurality of electrode icons corresponding to the plurality of electrodes,
wherein the processor is configured to determine, based on the impedance data, one or more impedance state electrodes from the plurality of electrodes that have an impedance state, and
wherein the processor is configured to provide on the display an impedance status representation for one or more of the plurality of electrode icons corresponding to the one or more impedance state electrodes.

2. The system of claim 1, wherein the processor is configured to provide on the display a therapy programming display screen, and receive user input via the therapy programming display screen to program an electrical therapy including to select active ones of the plurality of electrodes for use to deliver the electrical therapy, wherein the impedance status representation is on the therapy display screen used to program the electrical therapy.

3. The system of claim 2, wherein the impedance status representation includes an “X” on or near the one or more of the plurality of icons corresponding to the one or more impedance state electrodes.

4. The system of claim 1, wherein the impedance status representation on the display does not include an impedance measurement.

5. The system of claim 1, wherein the one or more impedance state electrodes have an out-of-bounds impedance.

6. The system of claim 1, wherein the one or more impedance state electrodes have a valid impedance.

7. The system of claim 1, wherein the plurality of electrodes is on a lead, and wherein the processor is configured to provide on the display a representation of the lead with the plurality of electrode icons.

8. The system of claim 7, wherein each of the electrode icons is labeled with a corresponding electrode number.

9. The system of claim 1, wherein the impedance status representation includes a color identifying the one or more impedance state electrodes.

10. The system of claim 1, wherein the impedance status representation includes a line segment under the one or more of the plurality of icons corresponding to the one or more impedance state electrodes.

11. The system of claim 1, wherein the impedance status representation includes an “X” on or near the one or more of the plurality of icons corresponding to the one or more impedance state electrodes.

12. The system of claim 1, wherein:
the impedance status representation is a first impedance status representation;
the processor is configured to determine, based on the impedance data, one or more second impedance state electrodes from the plurality of electrodes, and
the processor is configured to provide on the display a second impedance status representation for one or more of the plurality of electrode icons corresponding to the one or more second impedance state electrodes.

13. The system of claim 1, wherein the medical device is configured deliver electrical stimulation using the plurality of electrodes.

14. The system of claim 1, wherein the medical device is configured to sense electrical signals using the plurality of electrodes.

15. The system of claim 1, wherein the processor is configured to provide on the display an impedance detail icon, and to respond to selection of the impedance detail icon by displaying in a separate window the measured impedances for the plurality of electrodes.

16. The system of claim 15, wherein the processor is configured to provide on the display a date and time when the impedance was measured.

17. The system of claim 1, wherein:
the plurality of electrodes is on a lead;
the processor is configured to provide on the display a representation of the lead with the plurality of electrode icons;
each of the electrode icons are labeled with a corresponding electrode number; and
the impedance status representation for the one or more of the plurality of icons includes at least one of:
a line segment under the one or more of the plurality of icons corresponding to the one or more impedance state electrodes;
an “X” on or near the one or more of the plurality of icons corresponding to the one or more impedance state electrodes; or
a color associated with the one or more of the plurality of icons corresponding to the one or more impedance state electrodes.

18. The system of claim 17, wherein the one or more impedance state electrodes have an out-of-bounds impedance, and the impedance status representation for the one or more of the plurality of icons indicates that the corresponding one or more impedance electrodes have the out-of-bounds impedance.

19. The system of claim 17, wherein the one or more impedance state electrodes have a valid impedance, and the impedance status representation for the one or more of the plurality of icons indicates that the corresponding one or more impedance electrodes has the valid impedance.

20. The system of claim 17, wherein:
the one or more impedance state electrodes include one or more first impedance state electrodes having an out-of-bounds impedance and one or more second impedance state electrodes having a valid impedance; and
the impedance status representation includes a first impedance status representation indicating that the corresponding one or more first impedance state electrodes have the out-of-bounds impedance, and a second impedance status representation indicating that the corresponding one or more of the second impedance state electrodes have the valid impedance.
